(19) United States
(12) Patent Application Publication
(10) Pub. No.: US 2007/0100157 A1
(43) Pub. Date: May 3, 2007

(54) Zeolite based catalytic process for preparation of acylated aromatic ethers

(75) Inventors: Raksh Vir Jasra, Gujarat (IN); Sengodagounder Muthusamy, Gujarat (IN); Yogiraj Mansukhlal Badheka, Gujarat (IN)

Correspondence Address: MARSHALL, GERSTEIN & BORUN LLP, 233 S. WACKER DRIVE, SUITE 6300, SEARS TOWER, CHICAGO, IL, 60606, US

(73) Assignee: COUNCIL OF SCIENTIFIC AND INDUSTRIAL RESEARCH, Geelong (AU)

(21) Appl. No.: 11/523,455

(22) Filed: Sep. 19, 2006

Related U.S. Application Data
(63) Continuation of application No. 10/910,677, filed on Aug. 3, 2004, now abandoned.

(51) Int. Cl. C07C 69/02

(52) U.S. Cl. 560/144

(57) ABSTRACT

The present invention relates to an improved zeolite based catalytic process for the preparation of acylated aromatic ethers and more particularly, the invention relates to catalysed acylation of anisole (methoxybenzene) and veratrole (1,2-dimethoxybenzene) for the preparation of acylated aromatic ether, namely, p-methoxyacetophenone and 3,4 dimethoxyacetophenone respectively using microporous alumino-silicates solids like zeolites.

### FIELD OF THE INVENTION

The present invention relates to an improved zeolite based catalytic process for the preparaton of acylated aromatic ethers and more panicularly, the invention relates to catalysed acylation of anisole (methoxybenzene) and veratrole (1,2-dimethoxybenene) for the preparation of acylated aromatic ether, namely, p-methoxyacetophenone and 3,4-dimethoxyaoetophenone respectively using microporous alumino-silicates solids like zeolites.

### BACKGROUND AND PRIOR ART DESCRIPTION

The acylated aromatic ethers are of commercial importance in the fine chemicals industries, as many synthetic fragrances and pharmaceutical contains an acyl group, and these ether are useful intermediates. Acylated anisole is used for synthesis of 2-4Methxybenzoyl) benzoic acid, the sodium salt of which is used as sweetening agent. Similarly, acylatd veratrole is a synthon for preparation of vesnarinone 1-(3,4-Dimethoxybenzoy)4(1,2,3,4-tetrahydro-2-oxo6quinoliyl) piperazine which is a cardiotonic drug.

Conventional process for the acylation of aromatic compounds, including aromatic ethers mostly employs Lewis acids such as AlCl₃. Aromatic ethers are particulary acylated by us Friedel-Crafts acylation reaction.

Reference is made to U.S. Pat. No. (5,637,773, 1993: Jean-Roger Desmurs et al.) wherein it is disclosed that 40 millimoles of an aromatic substrates and 10 millimoles of acylating agents was mixed with Bismuth halide, preferably bismuth chloride at room temperture and then the reaction mixture was refluxed for 6 hours to give 67% of 4-acylated anisole. Such a process uses more tham stochiometric amounts of bismuth chloride and also poses problem of post reaction catalysts separation. Furthermore, the Lewis acid must be eliminated from the reaction medium by carrying out acidic or basic hydrolysis at the end of the reaction.

Reference is made to German patent (DE 3809260, 1989, Botta A., et al.) wherein anisole and acetic anhydride were stirred for 3 hours with Mordenite zeolite catalyst 160° C. under 20 bar of nitrogen pressure to give 75% conversion with 98% selectivity for p-methoxyactophenone.

This process has disadvantage of operating at high temperature and very high pressure and also needs a solvent.

Reference is made to Japanese patent (93-317557, 19931217, CAN124:8397 Myata, Akira et al.) wherein a mixture of veratrole and propionyl chloride, in the presence of Zeolite-β was refluxed for three hours to give 3,4-dimethoxypropiophenone. The drawback of the process is that it uses propionyl chloride as an acylating agent which generates toxic hydrochloric acid during acylation reaction. After 3 hours it gives 67.8% of dimethoxypropiophenone. Reference is made to the work of C. Kuroda et al., (Sci. Papas InstPhys. Chem. Res. 18, pp SI-60 (1932)) which describes the preparation of methoxyacetophenones by reaction of an aromatic compound carrying methoxy group with acetyl chloride in the presence of excess amount of AlCl₃ to obtain high conversions. Due to complexation of AlCl₃ with ketone formed during the reaction, more than stochiometric amounts of aluminum chloride is used. Use of corrosive and irritant AlCl₃ and need of post reaction effluent treatment process are some of the disadvantages of the process. The major drawback of the above stated process is separation of catalysts after completion of the reaction. This necessitates a long, expensive treatment following hydrolysis, extraction of the organic phase, separation of organic and aqueous phase and even dying of latter. Further, there are problems with aqueous saline effluent which has to be neutralized and which necessities additional operation. Also, the Lewis acid used during the reaction cannot be recycled, as it has been hydrolyzed Reference is made to the work of H. Burton and P.F.G.Praill (Journal of Chemical Society, Apr.-1950, pp1203-1206) wherein it is reported that Acetyl Perchlorate formed in-situ from Silver Perchlorates and acetyl chloride, is an effective acylating agent and will convert anisole into p-methyoxytophenone in about 70% yield. However, this process has disadvantages of using perchlortes which is a hazardous chemical.

Reference is made to the work of E.J., Bourne, et al. (Journal of Chemical Society, Mar.-1951, pp718-720) wherein trifluoroacetic anhydride catalyst is shown to promote the condensation between suitably activated aromatic compound and carboxylic acid or sulphonic acid to give ketones or sulphonea, respectively. In this process, the reaction was carried out at room temperature. According to Boure, et al., Anisole was added to a mixture of trifluroacetc anhydride and acetic acid. After three hours the solution was poured into excess sodium hydrogen carbonate solution and then extracted with chlorofom. The extract was dried, filtered and evaporated to obtain dry syrup. The dry syrup was crystallized to obtain 78% of p-methoxyacetophenone.

The reported process is multi-step process wherein separation of the product with very high recovery is a limitation.

Reference is made to the work of Cullinane N. M. et al. (Journal of Chemical Society, Feb.-1952, pp-376-380) wherein acylation of benzene, toluene and anisole is reported using TiCl₄ as a catalyst acids, acid chlorides and anhydride were used as the acylating agents. According to Cullinane et al., Acid is least and anhydride is most effective for the formation of acylated products. When anisole (0.15 g-mols), acetic anhydride (0.1 g-mol) and titanium tetrachloride (0.22 g-mol) are used, the yield of p-acylated product was reported to be 76% after 4 hours. When anisole (0.2 g-mols), benzoic acid (0.2 g-mols) and titanium tertachloride (0.4 g-mols) are used, the yield of p-acylated product was reported to be 63% after 1.5 hours. However, the process has the disadvantage of involving post reaction effluent treatment.

Thus there is a need to provide an improved process for the acylation of aromatic ethers.

### OBJECTS OF THE PRESENT INVENTION

The main object of the present invention is to provide an improved zeolite based catalytic process for the preparation of acylated aromatic ethers, which obviates the drawbacks as detailed above.

Another object of the present invention is to develop crystalline micro porous alumino-silicate catalyst based acylation process for aromatic ethers, which operates at moderate conditions of pressure, temperature and without the requirement of any solvent and yields high conversions for verntaole and anisole.

Yet another object of the present invention is to provide a process for acylation of aromatic ethers using solid acid heterogeneous catalysts, which are enviromentally friendly and safe in handling.

Still another object of the present invention is to provide a process for acylation of aromatic ether selectively at para position.

A further object of the present invention is to provide a process for acylation of aromatic ethers wherein the acylating agent used does not generate any hazardous by-product.

A further more object of the present invention is to provide a process where acylation of aromatic ether is carried out catalytically with high atom economy without giving rise to by-products.

### DETAILED DESCRIPTION OF THE PRESENT INVENTION

Accordingly, the present invention provides an improved zeolite based catalytic process for the production of acylated aromatic ethers, which comprises the steps of:
a) reacting an aromatic ether selected from a group comprising of anisole and veratrole with an acylating agent at temperature in the range of 80-120° C. for time period in the range of 1 to 8 hours without using any solvent, in the presence of rare earth exchanged zeolite catalyst having general formula:
   M₂/nO, Al₂O₃, xSiO₂, wH₂O
wherein: M is an alkali and/or lanthanide cation or proton; n is the valency of the metal M which may be alkali and/or lanthanide cations or protons; x is the SiO₂/Al₂O₃ ratio which may vary from 3 to 24 and W is the weight percentage of water adsorbed in the pores which may vary from 1 to 20% depending upon the relative humidity, temperature and zeolite type; Si/Al ratio is in the range of 5.5 to 20; aromatic ether to catalyst ratio is in the range of 1:3 to 1:5; to form an acylated aromatic ether in reaction mixture;
b) separating the zeolite catalyst from the reaction mixture of step (a); and
c) separating the acylated aromatic ether from the mixture of step (b).

In an embodiment of the present invention, the acylated aromatic ether obtained is p-acyl veratrole when the aromatic ether used is veratrole.

In another embodiment of the present invention, the acylated aromatic ether obtained is p-acyl anisole when the aromatic ether used is anisole.

In yet another embodiment of the present invention, the zeolite catalyst used is selected from the group comprising of Zeolite-Y, Zeolite-B, mordenite, and ZSM-5.

In still another embodiment of the present invention, the zeolite catalyst used has crystallinity in the range of 65 to 99%.

In one more embodiment of the present invention, the amount of rare earth metals incorporated in the zeolite catalyst is in the range of 10 to 3% by wt.

In one another embodiment of the present invention, the rare earth metals incorporated in the zeolite are selected from lanthanum, cerium, neodymium, prasedymium and samarium.

In a further embodiment of the present invention the acylating agent used is a chloride or anhydride of acetic acid.

In a further more embodiment of the present invention, where in step (c), the acylated aromatic ether is separated from the reaction mixture by distillation.

In a typical procedure for the preparation of the catalyst, Zeolite Y or β in its sodium form was taken and further treated in following two steps a) conversion to H-form and b) conversion of H-from to lanthanides form. For preparing H-form of the Zeolite, 10 grams of sodium zeolite was refluxed with 100 ml of 1M NH₄Cl solution for 6 hours at 80 to 110° C. The system was cooled following which solid catalyst was filtered, washed with hot distilled water till the filtrate became chloride free as tested by AgNO₃ and dried in an oven overnight at 110° C. for removing of the moisture. Thus dried sample was calcined at 550° C. for 6-10 hours to get H form of zeolite. The catalyst thus obtained was cooled at ambient temperature.

In a typical procedure for the preparation of final zeolite catalysts in lanthanide form, 10 grams of the above prepared zeolite in its H-form was refluxed with 100 ml of the 0.01 M solution of soluble salt like nitrate, chloride, or acetate of relative lanthanide cations for 6 to 10 hours at 80 to 100° C. Then the catalyst was filtered after cooling the contents, washed with hot distilled water till the filtrate became anion free and dried in an oven overnight at 110° C. for removing of the moisture. Activation of these prepared catalysts was done at 400° C. in air for 4 to 10 hours prior to catalytic studies.

The zeolites prepared were characterized for crystallinity by using X-ray powder diffraction using Philips X'perts MPD model and for BET surface area using Micromeritics ASAP-surface area analyzers.

Catalytic studies using above catalysts were done in continuous stirred tank reactor (CSTR) of 50 ml capacity having temperature controller, water circulator, magnetic stirrer and moisture trap. Typically, 5.5 g of veratrole (or 4.3 g of anisole) and 3.5 g of acetic anhydride were taken in a 50 ml capacity round bottom flask to which 2 gm of the catalyst after activation at 400° C. for 4 to 10 hours in muffle furnace was added. The round bottom flask was fitted with a condenser through which constant temperature water was circulated. Moisture trap was attached at the end of the condenser. The contents of the flask were constantly stirred using a magnetic stirrer. The flask was kept in an oil bath whose temperature was slowly raised to desired reaction temperature. The content of the flask were analyzed by gas chromatography at different time intervals ranging from 1 to 8 hours. The yield was followed over time by taking aliquots which were analyzed by Gas Chromatography HP model 6890 using capillary column HP-5. Percent yield of p-acyl anisole or p-acyl veratrole was calculated using following equation Percent Yield=number of moles of para acyl aromatic ether actually formed/number of moles of para acyl aromatic ether theoretically expected.

From these illustrations, it can be observed that zeolite based solid acid catalysts can be used both for anisole and veratrole acylation to produce selectively the desired p-acylated ether. Among all the zeolite studied, zeolite-β shows highest yields for both the compounds followed by zeolite Y. Zeolite ZSM-5 and mordenite show very poor activity for this conversion. This probably is due to smaller cavity size of these zeolites compared to Zeolite β and Y which offers diffusional resistance to acylated products. Among the various zeolite β studied, the one modified by incorporating cerium into it showed highest yields both for anisole and veratrole.

The inventive steps adopted in this invention are:
1. Zeolite-beta and zeolite-Y are modified with rare earth in the range of 10 to 30 weight % to make the catalysts more compatible with acylation reactions.
2. The acylation reaction is carried out in single step so that the multi-step process can be avoided.
3. The lower temperature and pressure favours the selectivity for para position, which is desired.
4. The catalytic reaction proceeds at relatively moderate temperature of 100° C. and at atmospheric pressure, which obviates the need of high temperature and pressure.
5. Acylation occurs without use of any solvent and without using hazardous and effluent generating acylating agent.

The following examples are given by way of illustration and therefore should not be construed to limit the scope of the present invention.

### EXAMPLE 1

10 grams of sodium zeolite Beta was refluxed with 100 ml of 1M aqueous solution of ammonium chloride for 6 hours at 80° C. This was followed by filtration, washing with hot distilled water till the filtrate became chloride free as checked by silver nitrate solution. Solid thus obtained was dried over night at 110° C. The dried sample was calcined at 550° C. in air for removing ammonia and the calcined solid was termed as zeolite H-Beta. 5.5 g of veratrole (or 4.3 g of anisole) and 3.5 g of acetic anhydride were taken in a 50 ml capacity round bottom flask to which 2 g of zeolite H-Beta as obtained above after activation at 400° C. for 4 hours in a muffle furnace was added. The round bottom flask was fitted with a condenser through which constant temperature water was circulated. Moisture trap was attached at the end of the condenser. The contents of the flask were constantly stirred using a magnetic stirrer. The flask was kept in an oil bath whose temperature was slowly raised to 100° C. The content of the flask were analyzed by gas chromatography at different time intervals ranging from 1 to 5 hours. The percent yield of p-acyl veratrole and p-acyl anisole obtained as shown in Table 1 varies from 30 to 88% and 24 to 47% respectively.

### EXAMPLE 2

10 g of zeolite H-Beta prepared by the process described in Example-1 was refluxed with 100 ml of 1M aqueous solution of Lanthanum nitrate for 6 hours at 80° C. This was followed by filtration, washing with hot distilled water till the filtrate became chloride free as checked by silver nitrate solution. Solid thus obtained was dried over night at 110° C. The dried sample was calcined at 550° C. in air and the calcined solid thus obtained was termed as zeolite La-Beta. Acylation of veratrole (or anisole) was carried out following the procedure as described in Example-1 using La-Beta as a catalyst instead of H-Beta. Same amounts of veratrole, acetic anhydride and catalyst were used. The percent yield of p-acyl veratrole and p-acyl anisole obtained as shown in Table 1 varies from 56 to 86% and 51-68% respectively.

### EXAMPLE 3

10 g of zeolite H-Beta prepared by the process described in Example-1 was refluxed with 100 ml of 1M aqueous solution of Cerium nitrate for 6 hours at 80° C. The Cerium nitrate solution used was prepared by treating cerium oxide with nitric acid). This was followed by filtration, washing with hot distilled water till the filtrate became chloride free as checked by silver nitrate solution. Solid thus obtained was dried over night at 100° C. The dried sample was calcined at 550° C. in air and the calcined solid was termed as zeolite Ce-Beta. Acylation of veratrole (or anisole) was carried out following the procedure as described in Example-1 using Ce-Beta as a catalyst instead of H-Beta. Same amounts of veratrole, acetic anhydride and catalyst were used. The percent yield of p-acyl veratrole and p-acyl anisole as shown in Table 1 from 60 to 93% and 39 to 65% respectively were obtained.

### EXAMPLE 4

10 g of zeolite H-Beta prepared by the process described in Example-1 was refluxed with 100 ml of 1M aqueous solution of Cerium nitrate for 6 hours at 80° C. This was followed by filtration, washing with hot distilled water till the filtrate became chloride free as checked by silver nitrate solution. Solid thus obtained was dried over night at 110° C. The dried sample was calcined at 550° C. in air and the calcined solid was termed as zeolite Ce-Beta. Acylation of veratrole (or anisole) was carried out following the procedure as described in Example-1 using Ce-Beta as a catalyst instead of H-Beta. Same amounts of veratrole, acetic anhydide and the catalyst were used however, the reaction was carried out at 80° C. The percent yield of p-acyl veratrole and p-acyl anisole obtained as shown in Table 1 varies from 29 to 38% and 14 to 23% and respectively.

### EXAMPLE 5

10 g of zeolite H-Beta prepared by the process described in Example-1 was refluxed with 100 ml of 1M aqueous solution of Cerium nitrate for 6 hours at 80° C. This was followed by filtration, washing with hot distilled water till the filtrate became chloride tree as checked by silver nitrate solution. Solid thus obtained was dried over night at 110° C. The dried sample was calcined at 550° C. in air and the calcined solid was termed as zeolite Ce-Beta. Acylation of veratrole (or 7 anisole) was carried out following the procedure as described Example-1 using Ce-Beta as a catalyst instead of H-Beta. Same amounts of veratrole or anisole, acetic anhydride and the catalyst were used however, the reaction was carried out at 120° C. The percent yield of p-acyl veratrole and p-acyl anisole obtained as shown in Table 1 varies from 28 to 55% and 8 to 16% to respectively.

### EXAMPLE 6

10 g of zeolite H-Beta prepared by the process described in Example-1 was refluxed with 100 ml of 1M aqueous solution of Cerium nitrate for 6 hours at 80° C. This was followed by filtration, washing with hot distilled water till the filtrate became chloride free as checked by silver nitrate solution. Solid thus obtained was dried over night at 110° C. The dried sample was calcined at 550° C. in air and the calcined solid was termed as zeolite Ce-Beta. Acylation of veratrole (or anisole) was carried out following the procedure as described in Example-1 using CeO as a catalyst instead of H-Beta. Same amounts of veratrole or anisole, acetic anhydride and the catalyst were used however, the reaction was carried at 150° C. The percent yield of p-acyl veratrole and p-acyl anisole obtained as shown in Table 1 varies from 22 to 53% and 8 to 13% respectively.

### EXAMPLE 7

10 g of zeolite H-Beta prepared by the process described in Example-1 was refluxed with 100 ml of 1M (aqueous solution of Neodymium nitrate for 6 hours at 80° C. This was followed by filtration, washing with hot distilled water till the filtrate became chloride free as checked by silver nitrate solution. Solid thus obtained was dried over night at 110° C. The dried sample was calcined at 550° C. in air and the calcined solid was termed as zeolite Nd-Beta. Acylation of veratrole (or anisole) was carried out following the procedure as described in Example-1 using Nd-Beta as a catalyst instead of H-Beta. Same amounts of veratoleor anisole, acetic anhydride and catalyst were used. The precent yield of p-acyl veratrole obtained as shown in Table 1 varies from 66 to 73% and 33 to 64% respectively.

### EXAMPLE 8

10 g of zeolite H-Beta prepared by the process described in Example-1 was refluxed with 100 ml of 1M aqueous solution of Praseodymium nitrate for 6 hours at 80° C. This was followed by filtration, washing with hot distilled water till the filtrate became chloride free as checked by silver nitrate solution. Solid thus obtained was dried over night at 110° C. The dried sample was calcined at 550° C. in air and the calcined solid was termed as zeolite Pr-Beta. Acylation of veratrole (or anisole) was carried out following the procedure as described in Example-1 Pr-Beta as a catalyst instead of H-Beta (same amounts of veratrole or anisoel, acetic anhydride and catalyst were used). The precent yield of p-acyl veratrole and p-acyl anisole obtained as shown in Table 1 varies from 39 to 89% and 20 to 33% respectively.

### EXAMPLE 9

10 g of zeolite H-Beta prepared as described in Example-1 was refluxed with 100 ml of 1M aqueous solution of Samarium chloride for 6 hours at 80° C. This was followed by filtration, washing with hot distilled water till the filtrate became chloride free as checked by silver nitrate solution. Solid thus obtained was dried over night at 110° C. The dried sample was calcined at 550° C. in air and calcined solid was termed as zeolite Sm-Beta. Acylation of veratrole (or anisole) was carried out following the procedure as described in Example-1 using Sm-Beta as a catalyst instead of H-Beta (same amounts of veratrole or anisole, acetic anhydride and catalyst were used). The percent yield of p-acyl veratrole and p-acyl anisole obtained as shown in Table 1 varies from 36 to 60% and 26 to 47% respectively.

### EXAMPLE 10

10 grams of zeolite, NaY, was refluxed with 100 ml of 1M aqueous solution of Ammonium chloride for 6 hours at 80° C. This was followed by filtration, washing with hot distilled water till the filtrate became chloride free as checked by silver nitrate solution. Solid thus obtained was dried over night at 110° C. The dried sample was calcined at 550° C. in air for removing ammonia and the calcined solid was termed as zeolite HY. 5.5 g of veratrole (or 4.3 g of anisole) and 3.5 g of acetic anhydride were taken in a 50 ml capacity round bottom flask to which 2 g of zeolite HY as obtained above after activation at 400° C. for 4 hours in muffle furnace was added. The round bottom flask was fitted with a condenser through which constant temperature water was circulated. Moisture trap was attached at the end of the condenser The contents of the flask were constantly stirred using a magnetic stirrer. The flask was kept in an oil bath whose temperature was slowly raised to 100° C. The content of the flask were analyzed by gas chromatography at different time intervals ranging from 1 to 5 hours. The percent yield of p-acyl veratrole and p-acyl anisole obtained as shown in Table 1 varies from 46 to 65% and 26 to 39% respectively.

### EXAMPLE 11

10 g of zeolite HY prepared by the process described in Example-10 was refluxed with 100 ml of 1M aqueous solution of Lanthanum nitrate for 6 hours at 80° C. This was followed by filtration, washing with hot distilled water till the filtrate became chloride free as checked by silver nitrate solution. Solid thus obtained was dried over night at 110° C. The dried sample was calcined at 550° C. in air and the calcined solid was termed as zeolite LaY. Acylation of veratrole (or anisole) was carried out following the procedure as described in Example-1 using LaY as a catalyst instead of Hβ (same amounts of veratrole, acetic anhydride and catalyst were used). The percent yield of p-acyl veratrole and p-acyl anisole as shown in Table 1 from 51 to 70 and 27 to 54% respectively were obtained.

### EXAMPLE 12

10 g of zeolite HY prepared by the process described in Example-10 was refluxed with 100 ml of 1M aqueous solution of Cerium nitrate for 6 hours at 80° C. This was followed by filtration, washing with hot distilled water till the filtrate became chloride free as checked by silver nitrate solution. Solid thus obtained was dried over night at 110° C. The dried sample was calcined at 550° C. in air and the calcined solid was termed as zeolite CeY. Acylation of veratrole (or anisole) was carried out following the procedure as described in Example-1 using CeY as a catalyst instead of H-Y. Same amounts of veratrole or anisole, acetic anhydride and catalyst were used. The percent yield of p-acyl veratrole and p-acyl anisole as shown in Table 1 from 56 to 79 and 34 and 62% respectively were obtained.

### EXAMPLE 13

10 g of zeolite HY prepared by the process described in Example 10 was refluxed with 100 ml of 1 M aqueous solution of Neodymium nitrate for 6 hours at 80° C. This was followed by filtration, washing with hot distilled water till the filtrate became chloride free as checked by silver nitrate solution. Solutions thus obtained was dried over night at 110° C. The dried sample was calcined at 550° C. in air and the calcined solid was termed as zeolite NdY. Acylation of veratrole (or anisole) was carried out following the procedure as described in Example-1 using NdY as a catalyst instead of H-Y (same amounts of veratrole or anisole, acetic anhydride and catalyst were used). The percent yield of p-acyl veratrole obtained as shown in Table 1 varies from 44 to 56 and 30 to 39% respectively.

### EXAMPLE 14

10 g of zeolite HY prepared by the process described in Example-10 was refluxed with 100 ml of 1M aqueous solution of Praseodymium nitrate for 6 hours at 8° C. This was followed by filtration, washing with hot distilled water till the filtrate became chloride free as checked by silver nitrate solution. Solid thus obtained was dried over night at 110° C. The dried sample was calcined at 550° C. in air and the calcined solid was termed as zeolite PrY. Acylation of veratrole (or anisole) was carried out following the procedure as described in Example-1 using Pr-Y as a catalyst instead of HY (same amounts of veratrole or anisole, acetic anhydride and catalyst were used). The percent yield of p-acyl veratrole and p-acyl anisole obtained as shown in Table 1 varies from 60 to 73% and 13 to 21% respectively.

### EXAMPLE 15

10 g of zeolite HY prepared by the process described in Example-10 was refluxed with 100 ml of 1M aqueous solution of Samarium nitrate for 6 hour at 80° C. This was followed by filtration, washing with hot distilled water till the filtrate became chloride free as checked by silver nitrate solution. Solid thus obtained was dried over night at 110° C. The dried sample was calcined at 550° C. in air and the calcined solid was termed as zeolite SmY. Acylation of veratrole (or anisole) was carried out following the procedure as described in Example-1 using SmY as a catalyst instead of H-Y (same amounts of veratrole or anisole, acetic anhydride and catalyst were used). The percent yield of p-acyl veratrole and p-acyl anisole obtained as shown in Table 1 varies from 39 to 61% and 7 to 35% respectively were obtained.

### EXAMPLE 16

10 g of zeolite sodium mordenite, NaM, was refluxed with 100 ml of 1M aqueous solution of ammonium chloride for 6 hours at 80° C. This was followed by filtration, washing with hot distilled water till the filtrate became chloride free as checked by silver nitrate solution. Solid thus obtained was dried over night at 110° C. The dried sample was calcined at 550° C. in air for removing ammonia and the calcined solid was termed as zeolite HM. 5.5 g of veratrole (or 4.3 g of anisole) and 3.5 g of acetic anhydride were taken in a 50 ml capacity round bottom flask to which 2 g of zeolite HM as obtained above after activation at 400° C. for 4 hours in muffle furnace was added. The round bottom flask was fitted with a condenser through which constant temperature water was circulated. Moisture trap was attached at the end of the condenser. The contents of the flask were constantly stirred using a magnetic stirrer. The flask was kept in an oil bath whose temperature was slowly raised to 100° C. The content of the flask were analyzed by gas chromatography at different time intervals ranging from 1 to 5 hours. The percent yield of p-acyl veratrole and p-acyl anisole obtained was less than 2% as shown in Table 1.

### EXAMPLE 17

10 grams of zeolite, NaSZM-5, was refluxed with 100 ml of 1M aqueous solution of ammonium chloride for 6 hours at 80° C. This was followed by filtration, washing with hot distilled water till the filtrate became chloride free as checked by silver nitrate solution. Solid thus obtained was dried over night at 110° C. The dried sample was calcined at 550° C. in air for removing ammonia and the calcined solid was termed as zeolite HZSM-5. 5.5 g of veratrole (or 43 g of anisole) and 3.5 g of acetic anhydride were taken in a 50 ml capacity round bottom flask to which 2 g of zeolite HZSM-5 as obtained above after activation at 400° C. for 4 hours in muffle furnace was added. The round bottom flask was fitted with a condenser through which constant temperature water was circulated. Moisture trap was attached at the end of the condenser. The contents of the flask were constantly stirred using a magnetic stirrer. The flask was kept in an oil bath whose temperature was slowly raised to 100° C. The content of the flask were analyzed by gas chromatography at different time intervals ranging from 1 to 5 hours. The percent yield of p-acyl veratrole and p-acyl anisole was less than 4% as shown in Table 1.

### EXAMPLE 18

Regeneration of the catalyst used in Example-3 was done using filtration, washing with polar solvent like acetone or ethyl alcohol followed by drying at 110° C. and activation at 400-500° C. The catalyst thus regenerated gave 90% and 88% yields for veratrole acylation under acylating conditions described in Example-3 after first and second regeneration.

The main advantages of the present inventions are:
1. Acylation is done without use of any solvent, i.e., it is solvent free synthesis single step reaction.
2. High atom utilization and low mass ratio of waste to desired product (E-factor) for these conversion reflecting the environmentally friendly production of p-acylated aromatic ether. Atom utilization is calculated by dividing the molecular weight of the desired product by the sum of the all substances produced in the stoichiometric equation, i.e. if we consider the acylation of the anisole and veratrole by acetic anhydride by using zeolite, reactions are represented as under. 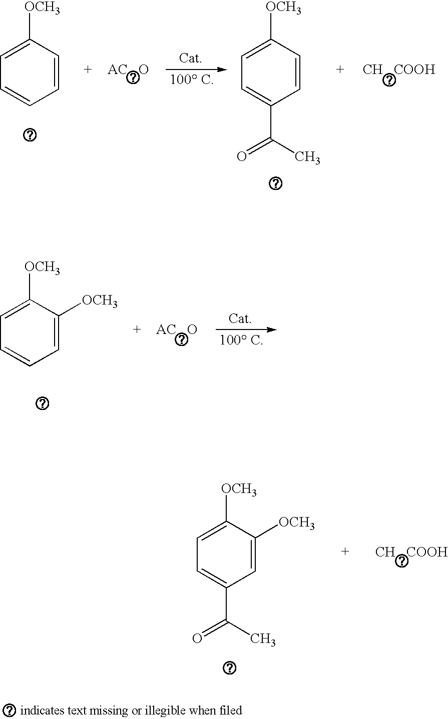
   Therefore in this reaction atom utilization is 151/210=71% for the acylation of anisole and 180/240=75% for the acylation of veratrole.
   E-factor is defined by the mass ratio of waste to desired product. In this reaction E-factor will be 60/151=0.4 for the acylaton for anisole and 60/80=0.33 for the acylation of veratrole.
3. Catalyst being solid in nature can be easily separated the reaction mixture by filtration or centrifuge.
4. Zeolites being highly crystalline and thermally stable can be regenerated by thermal treatment and re-used.
5. Shape selectivity towards para selectivity is observed in very high yield values for the products.

6. Zeolite based catalysts are easy in handling in comparison conventional Friedel-Craft acylation catalysts like H₂SO₄, HF, AlCl₃ and other Lewis acid.

TABLE 1

| | | Percent Yield different reaction time | | | | | |
|---|---|---|---|---|---|---|---|
| Example No. | Catalyst | 1 hr | 2 hr | 3 hr | 4 hr | 5 hr | 6 hr |

a. Percent yields of p-acyl veratrole obtained on acylation
using different catalysts

| | | | | | | | |
|---|---|---|---|---|---|---|---|
| 1. | Hβ | 30 | 33 | 52 | 88 | 79 | — |
| 2. | Laβ | 56 | 86 | 65 | 59 | — | — |
| 3. | Ceβ | 60 | 68 | 77 | 93 | 86 | |
| 4. | Ceβ | 29 | 29 | 38 | 32 | — | — |
| 5. | Ceβ | 28 | 35 | 58 | 55 | — | — |
| 6. | Ceβ | 22 | 28 | 53 | 48 | — | — |
| 7. | Ndβ | 66 | 68 | 70 | 73 | 68 | — |
| 8. | Pr-β | 39 | 42 | 54 | 71 | 89 | 86 |
| 9. | Smβ | 36 | 44 | 46 | 60 | 56 | — |
| 10. | HY | 46 | 49 | 60 | 65 | 56 | — |
| 11. | LaY | 51 | 62 | 70 | 59 | 55 | — |
| 12. | CeY | 56 | 60 | 76 | 79 | 69 | — |
| 13. | NdY | 44 | 52 | 56 | 55 | 48 | — |
| 14. | Pr-Y | 66 | 68 | 73 | 60 | | — |
| 15. | SmY | 39 | 52 | 61 | 59 | | — |
| 16. | HM | 0.41 | 0.52 | 1.23 | 0.97 | | — |
| 17. | HZSM-5 | 0.58 | 1.22 | 1.93 | 3.11 | 2.88 | — |

b. Percent yields of p-acyl anisole obtained on acylation of anisole
using different catalysts

| | | | | | | | |
|---|---|---|---|---|---|---|---|
| 1. | Hβ | 24 | 28 | 33 | 47 | 37 | — |
| 2. | Laβ | 51 | 65 | 68 | 59 | — | — |
| 3. | Ceβ | 39 | 42 | 65 | 61 | — | — |
| 4. | Ceβ | 14 | 23 | 18 | — | — | — |
| 5. | Ceβ | 8 | 12 | 16 | 13 | — | — |
| 6. | Ceβ | 8 | 13 | 18 | 14 | — | — |
| 7. | Ndβ | 33 | 42 | 58 | 60 | 64 | 45 |
| 8. | Prβ | 20 | 28 | 33 | 29 | — | — |
| 9. | Smβ | 26 | 35 | 47 | 38 | — | — |
| 10. | HY | 26 | 35 | 47 | 39 | — | — |
| 11. | LaY | 27 | 35 | 36 | 47 | 54 | 46 |
| 12. | CeY | 34 | 45 | 62 | 59 | — | — |
| 13. | NdY | 30 | 37 | 39 | 37 | — | — |
| 14. | PrY | 13 | 21 | 14 | 12 | — | — |
| 15. | SmY | 7 | 8 | 21 | 35 | 31 | — |
| 16. | HM | 0.21 | 0.32 | 0.85 | 0.91 | 0.87 | — |
| 17. | HZSM-5 | 0.62 | 0.78 | 1.48 | 1.23 | — | — |

1. A zeolite based catalytic process for the production of acylated aromatic ethers, said process comprising the steps of:—
a. reacting an aromatic ether selected from a group comprising of anisole and veratrole with an acylating agent at temperature in the range of 80-120° C. for time period in the range of 1 to 8 hours without using any solvent, in the presence of rare earth exchanged zeolite catalyst having general formula:
M₂/nO, Al₂O₃, xSiO₂, wH₂O
wherein: M is an alkali and/or lanthanide cation or proton; n is valency of M; x is in the SiO₂/Al₂O₃ ratio which is in the range of 3 to 24; W is the weight of water adsorbed which is in the range of 1 to 20% Si/Al ratio is in the range of 5.5 to 20; aromatic ether to catalyst ratio is in the range of 1:3 to 1:5;
to form an acylated aromatic ether in reaction mixture;
b. separating the zeolite catalyst from the reaction mixture of step (a); and
c. separating the acylated aromatic ether from the mixture of step (b).

2. A process as claimed in claim 1, wherein the acylated aromatic ether obtained is p-acyl veratrole when the aromatic ether used is veratrole.

3. A process as claimed in claim 1, wherein the acylated aromatic ether obtained is p-acyl anisole when the aromatic ether used is anisole.

4. A process as claimed in claim 1, wherein the zeolite catalyst used is selected from the group comprising of Zeolite-Y, Zeolite-B, mordenite, and ZSM-5.

5. A process as claimed in claim 1, wherein the zeolite catalyst used has crystallinity in the range of 65 to 99%.

6. A process as claimed in claim 1, wherein the amount of rare earth metals incorporated in the zeolite catalyst is in the range of 10 to 300% by wt.

7. A process as claimed in claim 1, wherein the rare earth metals incorporated in the zeolite are selected from lanthanum, cerium, neodymium, praseodymium and samarium.

8. A process as claimed in claim 1, wherein the acylating agent used is a chloride or anhydride of acetic acid.

9. A process as claimed in claim 1 wherein in step (c), the acylated aromatic ether is separated from the reaction mixture by distillation.

10. A process as claimed in claim 1, wherein about 93% yield of veratrole is obtained after 4 hours when Ce-β zeolite is used.

11. A process as claimed in claim 1, wherein about 65% yield of anisole is obtained after 3 hours when Ce-β zeolite is used.
